(19) United States
(12) Patent Application Publication
(10) Pub. No.: US 2026/0325312 A1
(43) Pub. Date: Oct. 22, 2026

(54) DIRECT COMPRESSION MOLDED PYROCARBON HUMERAL HEAD ASSEMBLY

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Monti Gourley, Austin, TX (US); Jacob Stegman, Austin, TX (US); Michael Seikel, Austin, TX (US); Bradley Jackson, Pflugerville, TX (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 18/997,953

(22) PCT Filed: Aug. 17, 2023
(86) PCT No.: PCT/US23/30428

§ 371 Date: Jan. 23, 2025

Related U.S. Application Data
(60) Provisional application No. 63/402,770, filed on Aug. 31, 2022.

(51) Int. Cl. A61F 2/40; A61F 2/30

(52) U.S. Cl. CPC A61F 2/4014; A61F 2002/30332; A61F 2002/30474; A61F 2002/30808; A61F 2002/4018; A61F 2002/4051; A61F 2310/00173

(57) ABSTRACT

An orthopedic humeral head implant for attachment to a bone member of an orthopedic joint such as, for example, a patient's humerus, is disclosed. In some examples, the implant may include a core (102) including a main body (104) and a flange (106) extending from the main body, and an interior element (108) formed over the core, wherein the interior element is made of a flexible polymeric material. The implant may further include a shell (116) formed over the interior element, wherein the shell includes a substrate (117) and an exterior layer (118) formed over the substrate, wherein the exterior layer is made of pyrocarbon.

### CROSS-REFERENCE TO RELATED APPLICATIONS

This is a non-provisional of, and claims the benefit of the filing date of, U.S. provisional patent application No. 63/402,770, filed Aug. 31, 2022, entitled “Direct Compression Molded Pyrocarbon Humeral Head Assembly,” the entirety of which application is incorporated by reference herein.

### FIELD OF THE DISCLOSURE

The present disclosure relates generally to orthopedic implants and, more particularly, to a pyrocarbon humeral head assembly formed by direct compression molding.

### BACKGROUND OF THE DISCLOSURE

One type of humeral head prostheses is a humeral head replacement implant. During humeral head replacement surgery, the entire humeral head is removed, and the humeral head replacement implant is attached to the humerus by a stem. Presently, many humeral head implants include a shell made of Co—Cr or Ti6Al4V alloys. However, it is recognized that these alloys are damaging to joint tissues. More recently, pyrocarbon shells have been used in place of Co—Cr or Ti6Al4V, as pyrocarbon has improved wear characteristics. However, pyrocarbon is a ceramic material, and is therefore brittle in nature, which contrasts with the ductile nature of metals present in other parts of the implant. Furthermore, in shoulder hemiarthroplasty, the humeral head implant is typically secured to the stem using a taper connection. However, the taper connection induces high hoop stresses and contact pressures, which increases the risk of fracture of the pyrocarbon shell.

Thus, it would be beneficial to provide an improved pyrocarbon humeral head implant assembly that meets the strength and performance requirements desired. It is with respect to these and other considerations that the present disclosure is provided.

### SUMMARY OF THE DISCLOSURE

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

In some examples, a humeral head assembly may include a pyrocarbon shell having an outer surface which is a section of a sphere, a concave interior surface, an open proximal base and a ridge extending circumferentially about a diameter of the shell adjacent the proximal base. The humeral head assembly may further include a metal substructure having a convex distal surface, means at a proximal end thereof for securing the implant in a cavity formed at the end of a bone, and a flange extending around an outer diameter of said metal substructure. The humeral head assembly may further include an intermediate element of polyethylene, characterized in that said polyethylene is direct compression molded to said metal substructure and said polyethylene is direct compression molded to the open proximal base of said pyrocarbon shell to create an fit that prevents its withdrawal from the subassembly of said pyrocarbon shell and said intermediate element, and in that said polyethylene, said metal substructure and said pyrocarbon shell engage each other at the ridge and at the flange, whereby forces at said joint are transferred compressively through said pyrocarbon shell to said convex surface of said metal substructure.

In any preceding or subsequent example, an implant for attachment to a bone member of an orthopedic joint may include a core comprising a main body and a flange extending from the main body, and an interior element formed over the core, wherein the interior element is made of a flexible polymeric material. The implant may further include a shell formed over the interior element, wherein the shell comprises a substrate and an exterior layer formed over the substrate, and wherein the exterior layer is made of pyrocarbon.

In any preceding or subsequent example, the main body of the core comprises an internal bore, and the flange of the core comprises a plurality of openings operable to receive the interior element.

In any preceding or subsequent example, the main body of the core further comprises an arcuate upper surface and a sidewall extending between the arcuate upper surface and the flange.

In any preceding or subsequent example, the main body of the core further comprises a plurality of channels extending between the arcuate upper surface and the sidewall, wherein the plurality of channels are operable to receive the interior element.

In any preceding or subsequent example, the interior element is made of ultra-high molecular weight polyethylene.

In any preceding or subsequent example, the substrate of the shell is made of graphite.

In any preceding or subsequent example, the substrate comprises an inner surface and an outer surface, wherein the inner surface comprises a plurality of protrusions in contact with the interior element.

In any preceding or subsequent example, the substrate further comprises an end surface extending between the inner surface and the outer surface, wherein the exterior layer is formed along the outer surface and the end surface.

In any preceding or subsequent example, the interior element is sandwiched between the flange of the core and the exterior layer formed on the end surface.

In any preceding or subsequent example, the implant further comprises an adapter coupled to the core, and a stem coupled to the adapter.

In any preceding or subsequent example, the adapter further comprises a male tapered trunnion coupleable with the core, and a split tapered shaft coupled with the stem.

In any preceding or subsequent example, the adapter further comprises a central flange between the male tapered trunnion and the split tapered shaft, wherein the central flange includes an opening to receive a tab of the stem.

In any preceding or subsequent example, the implant further comprises a fastener within a central bore of the adapter, wherein the fastener is coupleable with the stem.

A method of forming an implant for attachment to a bone member of an orthopedic joint may include forming a core comprising a main body and a flange extending from the main body, and forming an interior element over the core, wherein the interior element is made of a flexible polymeric material. The method may further include compression molding a shell onto the interior element, wherein the shell comprises a substrate and an exterior layer formed over the substrate, and wherein the exterior layer is made of pyrocarbon.

In any preceding or subsequent example, compression molding the shell onto the interior element causes the interior element to expand into a plurality of openings of the core.

In any preceding or subsequent example, the method may further comprise providing a plurality of channels through the core, wherein the plurality of channels extend between an arcuate upper surface and a sidewall of the core, and wherein compression molding the shell onto the interior element causes the interior element to expand into the plurality of channels.

In any preceding or subsequent example, the method may further comprise providing a plurality of protrusions along an inner surface of the substrate, wherein the plurality of protrusions are in contact with the interior element after the shell is compression molded onto the interior element.

In any preceding or subsequent example, the method may further comprise forming the exterior layer along an outer surface and an end surface of the substrate, wherein the end surface extends between the inner surface and the outer surface.

In any preceding or subsequent example, the method may further comprise coupling an adapter to the core, and coupling the adapter to a stem.

In any preceding or subsequent example, coupling the adapter to the core comprises inserting a male tapered trunnion of the adapter into an internal bore of the core, and coupling the adapter to the stem comprises inserting a split tapered shaft of the adapter into a central opening of the stem.

In any preceding or subsequent example, coupling the adapter to the stem further comprises inserting a tab of the stem into an opening of the core.

In any preceding or subsequent example, coupling the adapter to the stem further comprises coupling a fastener to the stem.

In any preceding or subsequent example, a direct compression molded pyrocarbon humeral head assembly may include a core comprising a main body and a flange extending from the main body, and an interior element formed over the core, wherein the interior element is made of a flexible polymeric material. The direct compression molded pyrocarbon humeral head assembly may further include a shell formed over the interior element, wherein the shell comprises a substrate and an exterior layer formed over the substrate, and wherein the exterior layer is made of pyrocarbon.

Examples of the present disclosure provide numerous advantages as will be further described herein. In accordance with one or more features of the present disclosure, a humeral head implant assembly is provided wherein a pyrocarbon exterior layer can be easily coupled to a metallic core. Further features and advantages of at least some of the examples of the present disclosure, as well as the structure and operation of various examples of the present disclosure, are described in detail below with reference to the accompanying drawings.

### BRIEF DESCRIPTION OF THE DRAWINGS

By way of examples of the disclosure will now be described, with reference to the accompanying drawings, in which:

FIG. 1A illustrates a bottom view of an example of a direct compression molded pyrocarbon implant assembly according to examples of the present disclosure;

FIG. 1B illustrates a side cross-sectional view of the direct compression molded pyrocarbon implant assembly according to examples of the present disclosure;

FIG. 1C illustrates an exploded side cross-sectional view of the direct compression molded pyrocarbon implant assembly according to examples of the present disclosure;

FIG. 2 illustrates a side cross-sectional view of an example of a direct compression molded pyrocarbon implant assembly according to examples of the present disclosure;

FIG. 3A illustrates a perspective view of a core of the direct compression molded pyrocarbon implant assembly according to examples of the present disclosure;

FIG. 3B illustrates a partial, side cross-sectional view of the core of the direct compression molded pyrocarbon implant assembly according to examples of the present disclosure;

FIG. 4A illustrates a perspective view of the core and an interior element of the direct compression molded pyrocarbon implant assembly according to examples of the present disclosure;

FIG. 4B illustrates a side cross-sectional view of the core and the interior element of the direct compression molded pyrocarbon implant assembly according to examples of the present disclosure;

FIG. 5A illustrates a perspective view of the interior element and a shell of the direct compression molded pyrocarbon implant assembly according to examples of the present disclosure;

FIG. 5B illustrates a partial, side cross-sectional view of the interior element and the shell of the direct compression molded pyrocarbon implant assembly according to examples of the present disclosure;

FIG. 6A illustrates a top view of a stem for use with the direct compression molded pyrocarbon implant assembly according to examples of the present disclosure;

FIG. 6B illustrates a perspective view of the stem for use with the direct compression molded pyrocarbon implant assembly according to examples of the present disclosure;

FIG. 7A illustrates a side view of an adapter for use with the direct compression molded pyrocarbon implant assembly according to examples of the present disclosure;

FIG. 7B illustrates a side cross-sectional view of the adapter for use with the direct compression molded pyrocarbon implant assembly according to examples of the present disclosure;

FIG. 7C illustrates a top view of the adapter for use with the direct compression molded pyrocarbon implant assembly according to examples of the present disclosure;

FIG. 7D illustrates a bottom view of the adapter for use with the direct compression molded pyrocarbon implant assembly according to examples of the present disclosure; and

FIG. 8 illustrates a fastener for use with the adapter and the direct compression molded pyrocarbon implant assembly according to examples of the present disclosure

The drawings are not necessarily to scale. The drawings are merely representations, not intended to portray specific parameters of the disclosure. The drawings are intended to depict various examples of the disclosure, and therefore are not considered as limiting in scope. In the drawings, like numbering represents like elements.

Furthermore, certain elements in some of the figures may be omitted, or illustrated not-to-scale, for illustrative clarity. The cross-sectional views may be in the form of “slices”, or “near-sighted” cross-sectional views, omitting certain background lines otherwise visible in a “true” cross-sectional view, for illustrative clarity. Furthermore, for clarity, some reference numbers may be omitted in certain drawings.

### DETAILED DESCRIPTION

An implant, assembly, and method in accordance with the present disclosure will now be described more fully hereinafter with reference to the accompanying drawings, where examples of the disclosure are shown. The implant, assembly, and method may be embodied in many different forms and are not to be construed as being limited to the examples set forth herein. Instead, these examples are provided so this disclosure will be thorough and complete, and will fully convey the scope of the implant, assembly, and method to those skilled in the art.

As will be described herein, examples of the present disclosure provide a pyrocarbon humeral head implant assembly (hereinafter “implant” or “assembly”) for attachment to a bone member of an orthopedic joint, such as a humerus of a shoulder joint. The implant described herein is designed to meet ASTM F 1378, Standard Specification for Shoulder Prostheses, which states that the normal joint reaction force acting on a humeral head is 2× an average body weight (e.g., approximately 180 lbs.). The joint force can be accommodated by designs described hereinafter.

The implant may include a core having a main body and a flange extending from the main body, wherein the core is coupled to the humerus by a stem. In some examples, the core is coupled to the stem by an adapter. The implant may further include an interior element formed over the core, wherein the interior element is made of a flexible polymeric material, such as polyethylene or other suitable biocompatible polymer or copolymer (e.g., ultra-high molecular weight polyethylene (UHMWPE)). A shell may then be formed over the interior element, wherein the shell comprises a substrate and an exterior layer formed over the substrate. The exterior layer may be made of pyrocarbon.

In some examples, to form the implant, the interior element is direct compression molded to the metal core. The shell is then direct compression molded to the core and the interior element. In direct compression molding, the material (e.g., UHMWPE) of the interior element starts in pellet form, and is then heated and formed to a specific shape using a tooling mold. The result is virgin UHMWPE in a specific desired shape. In examples of the present disclosure, the UHMWPE is direct compression molded a second time, not by a tooling mold, but by the shell when the shell is compressed onto the UHMWPE. The UHMWPE expands, flows, and fills the space(s) of the concave shell and adheres to both the shell and the core creating the assembly.

In some examples, the UHMWPE may be direct compression molded into the shell first, and the core may then be direct compression molded onto the shell.

In some examples, for increased strength in the connection of the core to the UHMWPE, the core includes a plurality of channels or tunnels extending between the top of the core and the perimeter of the core. The UHMWPE fills the tunnels during the direct compression molding, creating loops that hold the UHMWPE to the core.

In some examples, the flange of the core extends past the cavity of the shell. The flange provides stability to the shell by preventing the shell from tilting.

The flange of the core may include a plurality of openings or counterbores of a shallow depth. These features increase the adhesion of the UHMWPE to the core, as the UHMWPE flows into the counterbores during direct compression molding.

In some examples, the shell may have a plurality of ribs on an interior concave surface. These ribs provide rotational stability of the shell, as the UHMWPE flows into the empty spaces around the ribs during direct compression molding.

In some examples, the shell has an undercut on the internal diameter of the shell. The UHMWPE fills the undercut during direct compression molding, thus increasing adhesion between the shell and the core. Furthermore, the UHMWPE flashes out between the core flange and the base of the pyrocarbon shell. The UHMWPE therefore contacts substantially all of the underside of the pyrocarbon shell, providing maximum support and load distribution. The UHMWPE prevents all contact of the Pyrocarbon shell to the metal core.

In some examples, an adapter may be used to connect the implant to the stem. The adapter may include a split taper, which connects the adapter to the stem. Although non-limiting, the split taper may be split twice with a thru hole down the center, defining four tapered tabs. These four tapered tabs slightly flex as they are impacted into the stem, producing a strong hold, but also assembling all the way down. A center screw may be placed into the thru hole and tightened into the stem, providing backup support to hold the implant adapter in place. The opposite side of the implant adapter may include a male tapered trunnion for connection with the humeral head.

The result is a strong implant construct with a reduced gap between the humeral head and the stem.

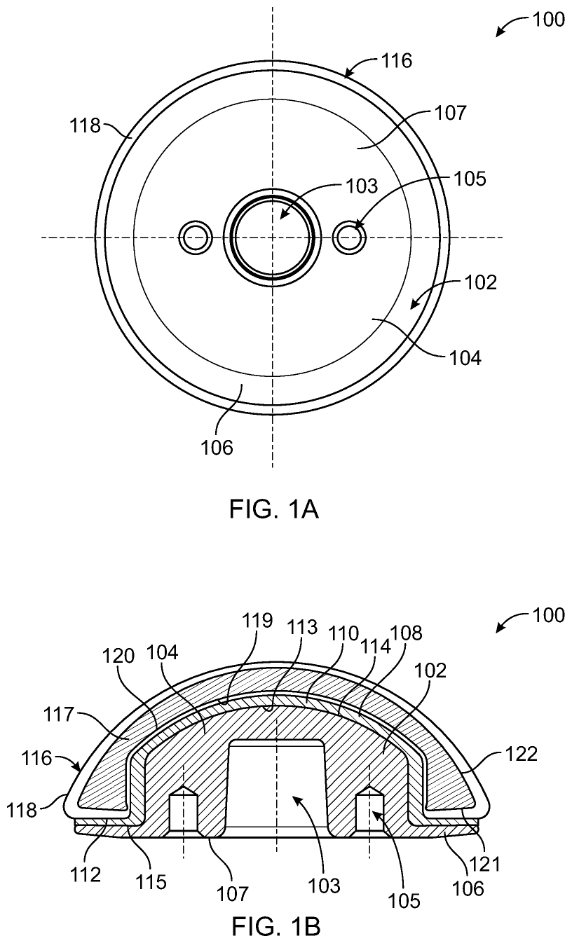

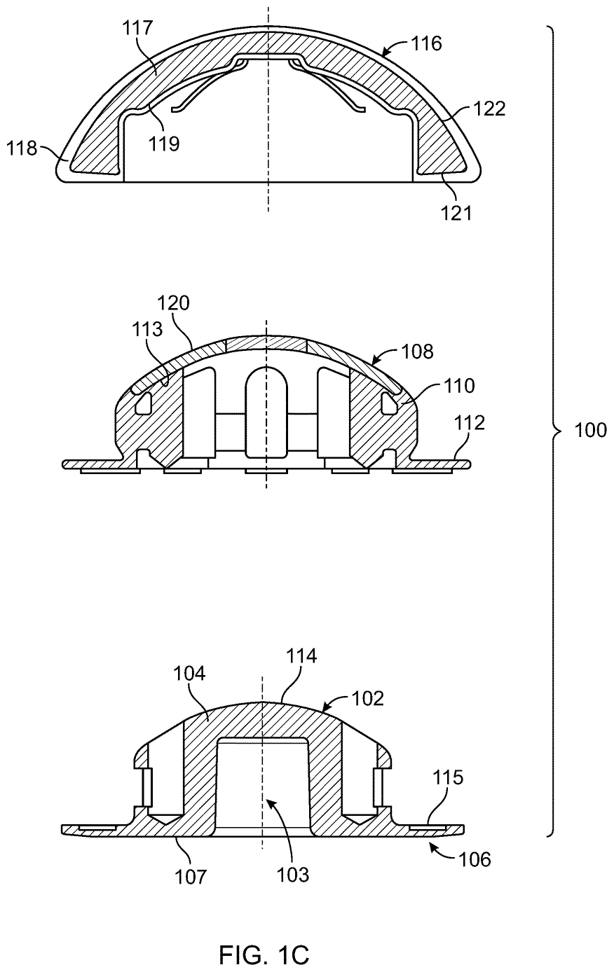

Turning now to FIGS. 1A-1C , a pyrocarbon humeral head assembly (hereinafter “assembly”) 100 according to examples of the present disclosure will be described. Although non-limiting, the assembly 100 may herein be described in the context of a shoulder hemiarthroplasty procedure in which the assembly is secured to a stem, which is secured to a humerus bone. The assembly 100 may include a core 102 having a main body 104 and a flange 106 extending from the main body 104. The core 102 may be a metal (e.g., titanium or CoCr) having an internal bore 103 and one or more openings 105 extending into a bottom surface 107. As will be described in greater detail below, the internal bore 103 and the openings 105 may receive features of a stem or an adapter coupled to a stem.

The assembly 100 may further include an interior element 108 formed over the core 102, wherein the interior element 108 is made of a flexible polymeric material, such as ultra-high molecular weight polyethylene (UHMWPE). The interior element 108 may include an arcuate body 110 and a flange 112 extending from the arcuate body 110. As shown, the interior element 108 generally conforms to the outer shape of the core 102, with an interior surface 113 of the interior element 108 being in direct contact with an upper surface 114 of the core 102. As further shown, the flange 112 of the interior element 108 may extend along an upper surface 115 of the flange 106 of the core 102.

A shell 116 may then be formed over the interior element 108, wherein the shell 116 includes a substrate 117 and an exterior layer 118 formed over the substrate 117. As shown, the substrate 117 and the exterior layer 118 may have a convex domed shape. In some examples, the exterior layer 118 is made entirely or partially of pyrocarbon, and the substrate 117 is made entirely or partially of graphite. The pyrocarbon exterior layer 118 constitutes an articular load-bearing surface of the assembly 100 and is designed to transmit loads in compression and not in tension due to the properties of the pyrocarbon. In some examples, the exterior layer 118 may completely coat the substrate 117, including along an inner surface 119 thereof.

When assembled, the exterior layer 118 along the inner surface 119 of the substrate 117 may be in direct contact with an exterior surface 120 of the interior element 108. As shown, the substrate 117 further includes an end surface 121 extending between the inner surface 119 and an outer surface 122, wherein the exterior layer 118 is formed along the outer surface 122 and the end surface 121. As a result, the interior element 108 is sandwiched between the flange 106 of the core 102 and the exterior layer 118 formed on the end surface 121 of the substrate 117. Providing the interior element 108 in contact with the exterior layer 118 along the entire inner surface 119 of the substrate 117 ensures maximum support and load distribution, and prevents contact between the shell 116 and the core 102.

In some examples, the pyrocarbon exterior layer 118 may be formed by depositing a layer of pyrolytic carbon on the graphitic substrate 117 using a chemical vapor deposition (CVD) process. A fluidized bed coater may be used to apply the pyrolytic carbon coating to the substrate 117, wherein the substrate 117 is levitated in the fluidized bed, which insures a continuous pyrolytic carbon coating is deposited thereupon.

Although non-limiting, the graphite of the substrate 117 may be dense, isotropic, fine-grain graphite, such as Poco AXF-5Q Biomedical Grade Graphite having a density greater than about 1.75gm/cm³. The pyrolytic carbon may have a density between about 1.7 and about 2.1 gm/cm³ and a hardness of at least about 200 DPH.

Such dense pyrocarbon is both stiffer and more fracture-resistant than the underlying machined graphite substrate 117 and, due to the high-temperature pyrolytic coating process, the pyrocarbon adheres strongly to the exterior surface of the isotropic graphite substrate 117. The result is one of mechanical reinforcement that provides strength to the composite structure and provides an integral implant exhibiting an elasticity modulus very close to that of human bone, which is approximately 23 gigapascals (GPa).

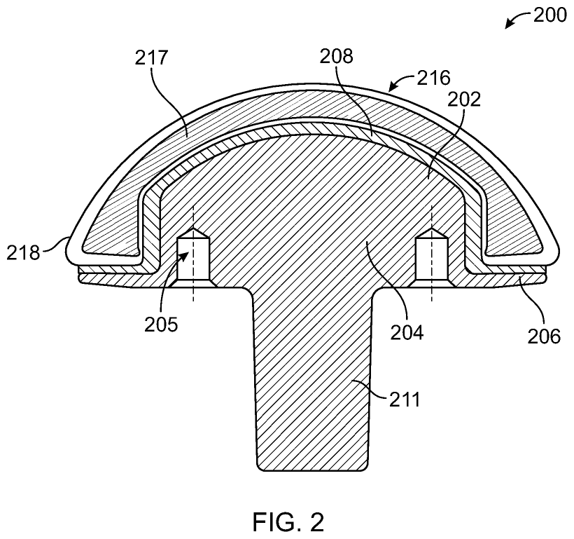

FIG. 2 demonstrates an alternative implant assembly (hereinafter “assembly”) 200 according to examples of the present disclosure. The assembly 200 may be the same or similar in many aspects to the assembly 100 described above. As such, only certain aspects of the assembly 200 will hereinafter be described for the sake of brevity. As shown, the assembly 200 may include a core 202 having a main body 204 and a flange 206 extending from the main body 204. The assembly 200 may further include an interior element 208 formed over the core 202, wherein the interior element 208 is made of UHMWPE. A shell 216 may then be formed over the interior element 208, wherein the shell includes a substrate 217 and an exterior layer 218 formed over the substrate 217. In some examples, the exterior layer 218 is made entirely or partially of pyrocarbon, and the substrate 217 is made entirely or partially of graphite.

In this example, the core 202 may include a tapered male trunnion 211 extending from the main body 204, the male trunnion 211 operable to be inserted into an opening of a stem. Openings 205 may receive tabs of the stem to provide additional stability to the connection.

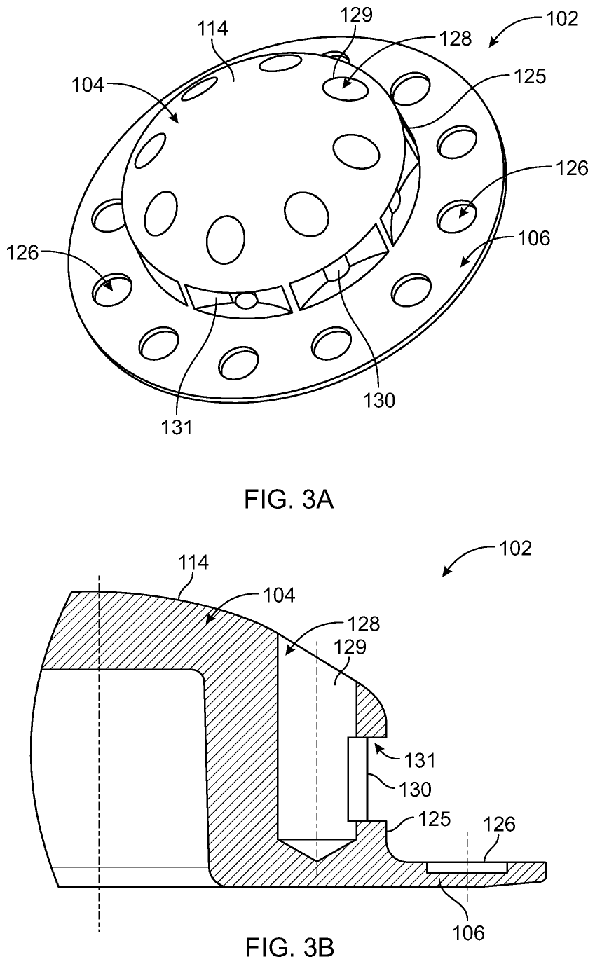

FIGS. 3A-3B demonstrate the core 102 in greater detail. As previously described, the core 102 includes the main body 104 and the flange 106 extending from the main body 104. The main body 104 may define the arcuate upper surface 114 and a sidewall 125, which extends between the upper surface 114 and the flange 106. The arcuate upper surface 114 may have convex domed shape. The flange 106 extends from a lower portion of the sidewall 125, and may include a plurality of openings 126 formed therein. In various examples, the openings 126 may extend entirely or partially through the flange 106. During formation of the assembly 100, the material of the interior element 108 flows into the openings 126 during a direct compression molding process to increase adhesion between the interior element 108 and the core 102. In some examples, the core 102 contains no openings, and instead sits entirely within the shell 116.

As further shown, the core 102 may include a plurality of channels 128 extending through the main body 104. More specifically, a first end 129 of each channel 128 may extend to the upper surface 114 of the main body 104 and a second end 130 of each channel 128 may extend to the sidewall 125. As further shown, the second end 130 of each channel 128 may be defined by a recessed section 131 of the sidewall 125. During the direct compression molding process, the material of the interior element 108 further flows into the channels 128 to increase adhesion between the interior element 108 and the core. In some cases, the material of the interior element 108 in the channels 128 connects with the material of the interior element 108 along the sidewall 125 to form a plurality of loops, which further reinforce the connection between the interior element and the core 102.

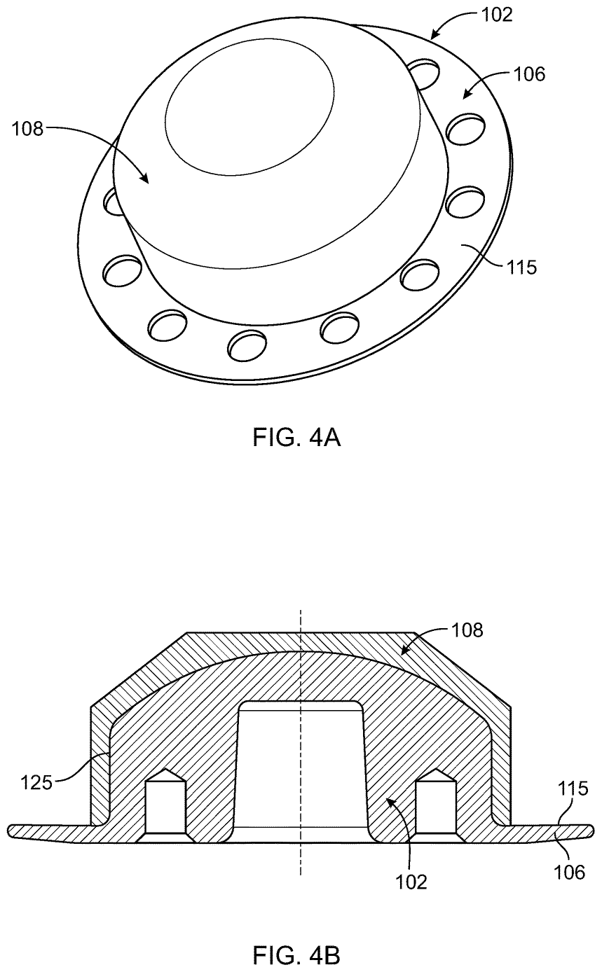

FIGS. 4A-4B demonstrate application of the interior element 108 to the core 102. The interior element 108 may initially be heated and formed to a specific shape, such as the general shape of the main body 104, using a tooling mold. In some examples, the interior element 108 may extend along the sidewall 125 to the upper surface 115 of the flange 106. In some examples, an adhesive or grouting agent (not shown) may be used between the interior element 108 and the core 102. Once the shell 116 is compressed atop the interior element 108 and the core 102, the material of the interior element 108 flashes out between the core 102 and the base of shell 116 to cover the upper surface 115 of the flange 106. In an alternative example, the interior element 108 could be manufactured by injection molding rather than direct compression molding. In an alternative example, the assembly 100 could be manufactured without interior element 108 but by direct metal laser sintering of the core 102 inside the shell 116. In an alternative example, the assembly 100 could be manufactured using an adhesive, which directly connects the core 102 and the shell 116.

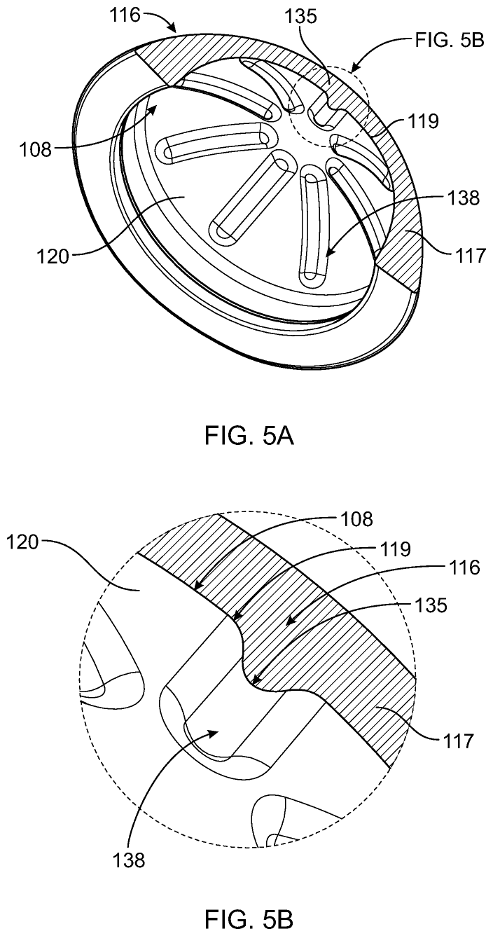

FIGS. 5A-5B demonstrate connection between the interior element 108 and the shell 116 in greater detail. In some examples, the inner surface 119 of the substrate 117 and/or the exterior layer 118 may include a plurality of ribs or protrusions 135 extending therefrom. In an alternative example, the inner surface 119 of the substrate 117 and/or the exterior layer 118 may include a plurality of indentations or recesses. In yet another example, the inner surface 119 of the substrate 117 and/or the exterior layer 118 may include a combination of indentations and recesses.

As shown, the protrusions 135 engage a corresponding set of recesses 138 formed in the exterior surface 120 of the interior element 108. As shown, the exterior surface 120 of the interior element 108 may have a convex domed shape. In some examples, the set of recesses 138 are formed when the material of the interior element 108 is initially heated and formed to the desired shape using the tooling mold. In other examples, the set of recesses 138 are formed as a result of direct compression molding when the shell 116 is applied to the interior element 108 and the core 102, which causes the material of the interior element 108 to flow into the empty spaces around the protrusions 135. In either case, the protrusions 135 and the recesses 138 engage one another to prevent or reduce rotation between the shell 116, the interior element 108, and the core 102. Once the shell 116 is secured to the interior element 108, the assembly 100 achieves compressive load transfer from the bearing surface of the exterior layer 118, through the interior element 108, and to the metal core 102.

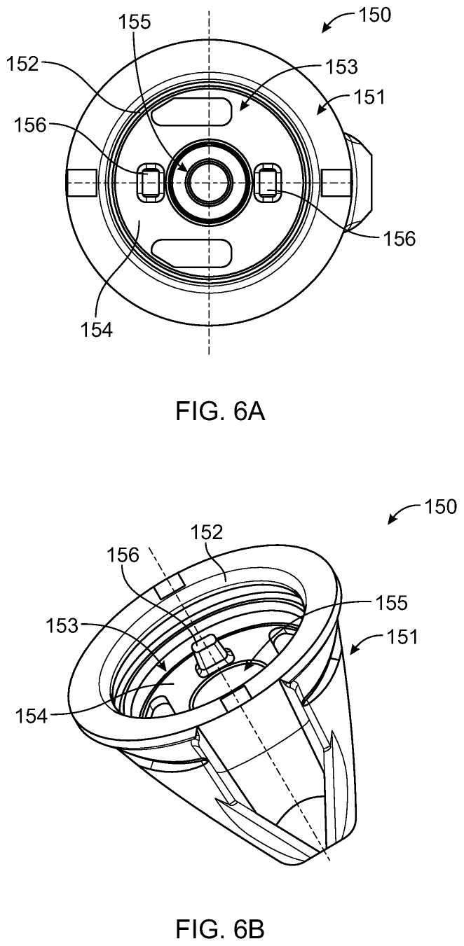

FIGS. 6A-6B demonstrate an example of a stem 150 operable to connect the assembly 100 to a bone member of an orthopedic joint, such as a humerus of a shoulder joint. As shown, the stem 150 may include a main body 151 having a circular wall 152, which defines a cavity 153. The stem 150 may further include an intermediate shelf 154 and a central opening 155 extending further into the main body 151. In some examples, one or more tabs 156 may extend from the intermediate shelf 154 for engagement with the assembly 100 or with an adapter, as will be further described below. In some examples, the assembly 100 and the stem 150 may be made modular to allow for assembly of different size head components with different size stem components to meet anatomic variations expected from one patient to another.

Furthermore, it will be appreciated that the stem 150 is only one possible stem example, and alternative stems may be used together with examples of the present disclosure.

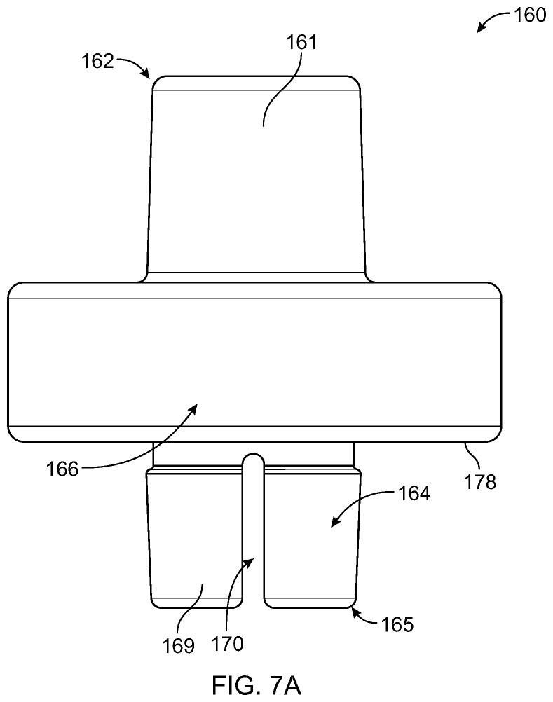

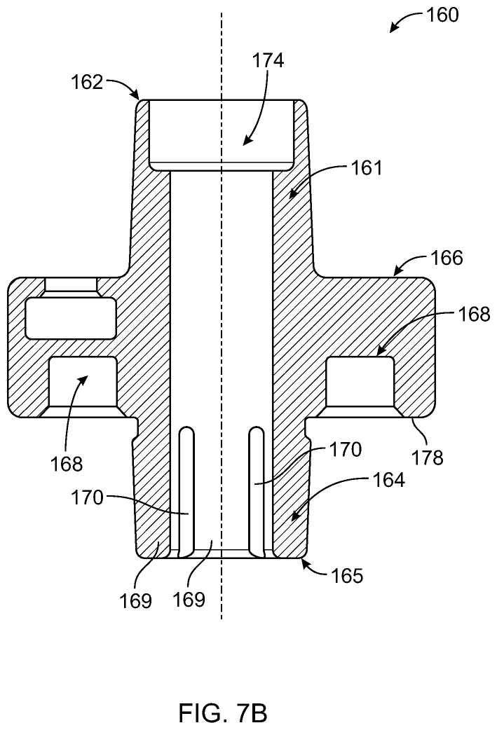

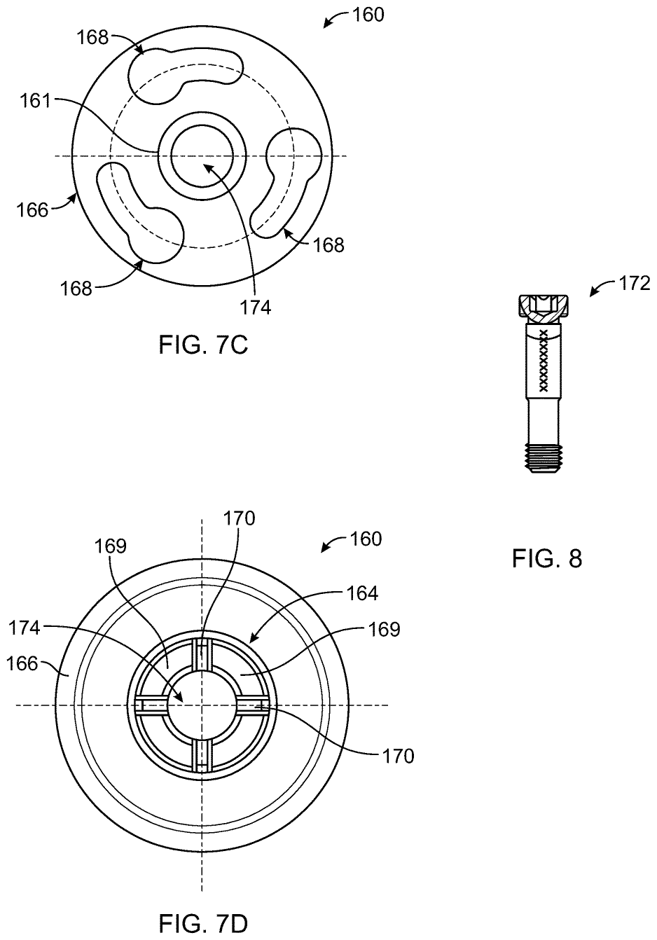

FIGS. 7A-7D demonstrate an adapter 160 according to examples of the present disclosure. The adapter 160 may be used to connect the core 102 to the stem 150. As shown, the adapter 160 may include a male tapered trunnion 161 at a first end 162, and a split tapered shaft 164 at a second end 165. The male tapered trunnion 161 is operable to extend within the internal bore 103 of the core 102, while the split tapered shaft 164 is operable to extend within the central opening 155 of the stem 150. Although non-limiting, the split tapered shaft 164 may include four tapered tabs 169 separated by a set of slits 170, the four tapered tabs 169 operable to flex and compress towards one another when engaged by the stem 150. The adapter 160 may further include a fastener (e.g., center screw) 172, as shown in FIG. 8, the fastener 172 extending within a central bore 174 of the adapter 160. During assembly, the fastener 172 may be inserted into the central opening 155 of the stem 150 to provide further stability between the adapter 160 and the stem 150. In some examples, the fastener 172 may include a straight or tapered shaft, which engages an interior surface of one or more of the four tapered tabs 169, e.g., by complementary threading, to provide additional locking strength.

In other examples, the connection features of the adapter 160 may be flipped, with the male tapered trunnion 161 being inserted within the central opening 155 of the stem 150 and the split tapered shaft 164 being inserted within the internal bore 103 of the core 102. In other examples, the male trunnion 161 and/or the tabs 169 may be implemented without a taper. In yet other examples, the second end 165 of the adapter 160 may include a UHMWPE snap ring in place of the split taper, the UHMWPE snap ring being operable to snap into the central opening 155 of the stem 150.

Between the first and second ends 162, 165 of the adapter 160 is a central flange 166. The central flange 166 may be seated within the cavity 153 of the main body 151 of the stem 150 when the stem 150 and the adapter 160 are coupled together. As shown, the central flange 166 may include a plurality of openings 168 operable to receive the tabs 156 of the stem 150. Once engaged, the tabs 156 prevent or limit rotation between the adapter 160 and the stem 150. In other examples, the tabs 156 are located along an underside 178 of the central flange 166 and the openings 158 are recessed into the intermediate shelf 154. Examples herein are not limited in this context.

In sum, examples of the present disclosure provide humeral head assemblies with improved compressive strength when compared to other pyrocarbon humeral head assemblies. The humeral head assemblies further provide improved axial tensile strength and improved torsional strength when compared to other pyrocarbon humeral head assemblies, as well as improved wear properties when compared to standard-of-care CoCr humeral head hemiarthroplasty devices.

Examples of the present disclosure provide numerous advantages. For example, the core includes a flange that extends past the cavity of the shell to provide stability to the shell by preventing the shell from tilting. In some prior art designs, the core does not include a flange, and therefore the shell is at risk of rotating in a fashion where the flat surface of the shell and the flat surface of the core are no longer parallel.

Furthermore, the flange of the core advantageously includes a plurality of counterbores, or openings, which increase the adhesion of the interior element to the core, as the material of the interior element expands or flows into the counterbores during direct compression molding.

Furthermore, the pyrocarbon shell advantageously includes a plurality of ribs along an underside. The ribs provide rotational stability of the shell, as the material of the interior element flows into the empty space around the ribs during direct compression molding. In some prior art designs, the concave interior surface of the shell is smooth, which allows the shell to freely spin on the core, resulting in particle generation during the joint replacement.

Furthermore, the pyrocarbon shell advantageously has an undercut on the internal diameter of the shell. The material of the interior element fills the undercut during direct compression molding, which better holds the shell to core.

Furthermore, the interior element flashes out between the core flange and the base of the pyrocarbon shell. The interior element therefore contacts the entire underside of the pyrocarbon shell, advantageously providing maximum support and load distribution. The interior element prevents all contact of the pyrocarbon shell to the metal core.

Furthermore, an adapter of the humeral head assembly may advantageously include a split taper that connects the implant adapter to the stem implant. The split taper may be split twice with a thru hole down the center, resulting in four tapered tabs. The tabs are designed to slightly flex towards one another as they are impacted into the stem, producing a strong hold, but also assembling to a desired depth. A center screw may be placed into the thru hole and tightened into the stem, providing backup support to hold the implant adapter in place. The opposite side of the implant adapter includes a male tapered trunnion that the humeral head can be connected to. The result is an implant construct that is strong while also reducing the gap between the humeral head and the stem.

The adapter advantageously allows the implant to be assembled to a precise height (i.e., all the way down), unlike traditional tapered connections, which result in a range of depths. Furthermore, the adapter includes multiple points of fixation, namely, the split taper and the center screw. The center screw provides more precise height control, with the additional strength advantage like those with the taper connection. By allowing the adapter and the implant to be recessed farther into the stem, additional stability is provided, as the lateral loading is distributed across the flange that is assembled flush, rather than only through the tapered trunnion. Furthermore, the adapter ensures that, once assembled, the assembly will not come apart, i.e., disassemble.

Furthermore, examples herein attach the pyrocarbon shell to the core in such a way to advantageously ensure that the pyrocarbon bearing surface is placed in compression when transmitting loads between the pyrocarbon shell and the core, thus minimizing damage to the pyrocarbon.

It should be understood that the various illustrated arrangements for mounting a domed pyrocarbon shell to a metal core is not limited to humeral head implants, and that similar designs can be used to attach pyrocarbon shells to metal substructures for substantially any orthopedic joint replacement, for example, a hip joint replacement.

While the present disclosure refers to certain examples, numerous modifications, alterations, and changes to the described examples are possible without departing from the sphere and scope of the present disclosure, as defined in the appended claim(s). Accordingly, it is intended that the present disclosure not be limited to the described examples, but that it has the full scope defined by the language of the following claims, and equivalents thereof. The discussion of any example is meant only to be explanatory and is not intended to suggest that the scope of the disclosure, including the claims, is limited to these examples. In other words, while illustrative examples of the disclosure have been described in detail herein, it is to be understood that the inventive concepts may be otherwise variously embodied and employed, and that the appended claims are intended to be construed to include such variations, except as limited by the prior art.

As used herein, an element or step recited in the singular and proceeded with the word “a” or “an” should be understood as not excluding plural elements or steps, unless such exclusion is explicitly recited. Furthermore, references to “one example” of the present disclosure are not intended to be interpreted as excluding the existence of additional examples that also incorporate the recited features.

The phrases “at least one”, “one or more”, and “and/or”, as used herein, are open-ended expressions that are both conjunctive and disjunctive in operation. The terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the present disclosure, and do not create limitations, particularly as to the position, orientation, or use of this disclosure.

Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. All rotational references describe relative movement between the various elements. Identification references (e.g., primary, secondary, first, second, third, fourth, etc.) are not intended to connote importance or priority but are used to distinguish one feature from another. The drawings are for purposes of illustration only and the dimensions, positions, order and relative to sizes reflected in the drawings attached hereto may vary.

The foregoing discussion has been presented for purposes of illustration and description and is not intended to limit the disclosure to the form or forms disclosed herein. For example, various features of the disclosure are grouped together in one or more example or configurations for the purpose of streamlining the disclosure. However, it should be understood that various features of the certain examples or configurations of the disclosure may be combined in alternate examples, or configurations. Any example or feature of any section, portion, or any other component shown or particularly described in relation to various examples of similar sections, portions, or components herein may be interchangeably applied to any other similar example or feature shown or described herein. Additionally, components with the same name may be the same or different, and one of ordinary skill in the art would understand each component could be modified in a similar fashion or substituted to perform the same function.

Moreover, the following claims are hereby incorporated into this Detailed Description by this reference, with each claim standing on its own as a separate example of the present disclosure.

What is claimed is:

1. An implant for attachment to a bone member of an orthopedic joint, the implant comprising:
a core comprising a main body and a flange extending from the main body;
an interior element formed over the core, wherein the interior element is formed over the main body of the core, and wherein the interior element is made of a flexible polymeric material;
a shell formed over the interior element, wherein the shell comprises a substrate and an exterior layer formed over the substrate, wherein the exterior layer is made of pyrocarbon.

2. The implant of claim 1, wherein the main body of the core comprises an internal bore, and wherein the flange of the core comprises a plurality of openings operable to receive the interior element.

3. The implant of claim 2, wherein the main body of the core further comprises:
an arcuate upper surface;
a sidewall extending between the arcuate upper surface and the flange.

4. The implant of claim 3, wherein the main body of the core further comprises a plurality of channels extending between the arcuate upper surface and the sidewall, and wherein the plurality of channels are operable to receive the interior element.

5. The implant of claim 1, wherein the interior element is made of ultra-high molecular weight polyethylene.

6. The implant of claim 1, wherein the substrate of the shell is made of graphite.

7. The implant of claim 1, wherein the substrate comprises an inner surface and an outer surface, and wherein the inner surface comprises a plurality of protrusions in contact with the interior element.

8. The implant of claim 7, wherein the substrate further comprises an end surface extending between the inner surface and the outer surface, and wherein the exterior layer is formed along the outer surface and the end surface.

9. The implant of claim 8, wherein the interior element is sandwiched between the flange of the core and the exterior layer formed on the end surface.

10. The implant of claim 1, further comprising:
an adapter coupled to the core; and
a stem coupled to the adapter.

11. The implant of claim 10, wherein the adapter comprises:
a male tapered trunnion coupleable with the core; and
a split tapered shaft coupled with the stem.

12. The implant of claim 11, wherein the adapter further comprises a central flange between the male tapered trunnion and the split tapered shaft, wherein the central flange includes an opening to receive a tab of the stem.

13. The implant of claim 10, further comprising a fastener with a central bore of the adapter, wherein the fastener is coupleable with the stem.

14. A method of forming an implant for attachment to a bone member of an orthopedic joint, the method comprising:
forming a core comprising a main body and a flange extending from the main body;
forming an interior element over the core, wherein the interior element is made of a flexible polymeric material;
compression molding a shell onto the interior element, wherein the shell comprises a substrate and an exterior layer formed over the substrate, wherein the exterior layer is made of pyrocarbon.

15. The method of claim 14, wherein compression molding the shell onto the interior element causes the interior element to expand into a plurality of openings of the core.

16. The method of claim 14, further comprising providing a plurality of channels through the core, wherein the plurality of channels extend between an arcuate upper surface and a sidewall, and wherein compression molding the shell onto the interior element causes the interior element to expand into the plurality of channels.

17. The method of claim 14, further comprising providing a plurality of protrusions along an inner surface of the substrate, wherein the plurality of protrusions are in contact with the interior element after the shell is compression molded onto the interior element.

18. The method of claim 17, further comprising forming the exterior layer along an outer surface and an end surface of the substrate, wherein the end surface extends between the inner surface and the outer surface.

19. The method of claim 14, further comprising:
coupling an adapter to the core; and
coupling the adapter to a stem.

20. The method of claim 19, wherein coupling the adapter to the core comprises inserting a male tapered trunnion of the adapter into an internal bore of the core, and wherein coupling the adapter to the stem comprises inserting a split tapered shaft of the adapter into a central opening of the stem.

21. The method of claim 19, wherein coupling the adapter to the stem further comprises inserting a tab of the stem into an opening of the core.

22. The method of claim 19, wherein coupling the adapter to the stem further comprises coupling a fastener to the stem.
